(19) United States
(12) Patent Application Publication
(10) Pub. No.: US 2005/0187537 A1
(43) Pub. Date: Aug. 25, 2005

(54) Angular deflection apparatus for use in confined spaces and method of use

(76) Inventors: Marvin P. Loeb, Huntington Beach, CA (US); Glenn D. Yeik, Lake Forest, CA (US); L. Dean Crawford, Irvine, CA (US)

Correspondence Address: GENE SCOTT; PATENT LAW & VENTURE GROUP, 3140 RED HILL AVENUE, SUITE 150, COSTA MESA, CA, 92626-3440, US

(21) Appl. No.: 10/783,574

(22) Filed: Feb. 19, 2004

(51) Int. Cl. A61B 17/00; A61N 2/00; A61B 18/18; A61B 18/04; A61F 7/00; A61F 7/12; A61B 17/32

(52) U.S. Cl. 606/1; 606/14; 606/27; 606/41; 606/49; 606/50; 607/99; 606/167

(57) ABSTRACT

A tube having a distal end portion made of a curved, flexible, shape retentive material such as superelastic, nickel-titanium memory metal alloy which has been heat-treated to retain a desired curved shape. The tube translates within and is constrained by a rigid sleeve, which may alternately be an instrument channel of an endoscope. When the distal end portion of the tube is extended from the sleeve, it returns to its original curved shape. Markings about the proximal and, or distal end portions of the tube enable the operator to know to what extent the distal end portion of the tube has been extended from the sleeve even when the distal end portion of the tube is not visible. The tube may be used for supporting and activating a cutting, abrading, coagulating, shrinking, or vaporizing device that is brought near or into contact with a tissue surface.

### BACKGROUND OF THE INVENTION

1. Incorporation by Reference

Applicant(s) hereby incorporate herein by reference, any and all U.S. patents, U.S. patent applications, and other documents and printed matter cited or referred to in this application.

2. Field of the Invention

This invention relates generally to apparatus for reaching confined and difficult areas with a work tool and more particularly to such an apparatus capable of manipulation to selected angles and positions when the therapeutic portion of the tool is fully or partially hidden from view.

3. Description of Related Art

The following defines the present state of this field:

Cutting, removing, coagulating or shrinking tissue in a confined space, such as in a: foraminal space in a spinal column, duct, surgically created passageway, or hollow organ, poses a significant difficulty to a surgeon. Such surgery is conducted using, for instance, a rotating burr, a shaver, or a radio frequency or laser energy emitter, and ideally is completed without damaging healthy, adjacent tissues. Access to such locations may be limited to specific angles of approach. The surgical instrument must have an outside diameter sufficiently small to enable it to be passed through a tiny opening such as a puncture or the instrument channel of an endoscope, for instance, which opening may be typically 3.8 mm across, and may be smaller.

One or more wires, extending through a flexible catheter and attached to its distal end, may be retracted to cause the distal end of the catheter to bend at a variety of angles. However, once the catheter is inserted into tissue, its sidewise movement is restricted, particularly if the tissue is relatively dense. Also, the angle at which it may be bent by retracting the wires may not be known by the operator, unless the device is observed by x-ray, MRI or other imaging means, which adds to the cost and complexity of the procedure.

It is an object of this invention to avoid the limitations of prior art devices by providing a device that is predictably maneuverable into a desired position so as to conduct the above listed surgical actions, and to perform other functions in confined spaces.

### SUMMARY OF THE INVENTION

The present invention teaches certain benefits in construction and use which give rise to the objectives described below.

A superelastic memory metal alloy of titanium and nickel can be heat treated to “remember” its heat-treated shape, regardless of how many times it is straightened out or bent. Such superelastic memory metal alloys, typically nickel-titanium alloys such as Nitinol® or NiTi® alloys, are sold by companies such as Memry Corporation of Bethel, Conn. and Shape Memory Applications, Inc, of San Jose, Calif.

Nickel-titanium alloys are relatively expensive. While the distal end portion of a tube, made entirely of nickel-titanium alloy, can be heat-treated to retain a curved shape, it is more practical to attach a short section of nickel-titanium alloy tubing to the distal end of a plastic tube, made of a material, such as PTFE, polyurethane or the like, or a metal tube made of a material, such as medical grade stainless steel. For example, about 1 to 6 cm, and more preferably about 2 to 4 cm of a tube made of a superelastic memory metal alloy of nickel and titanium in the proportion of about 55.8 Ni to 44.2 Ti, by weight, with an outside diameter of about 1 mm to 5 mm, and more preferably about 1.5 to 3.5 mm, can be heat-treated to retain a desired curve or bend at an angle of about 5° to 180°, and more preferably about 20° to 120°, with a bend radius of about 0.3 cm to 3 cm, and more preferably about 0.5 cm to 1.5 cm. This section of nickel-titanium alloy tubing can then be attached to the distal end of a plastic tube with an adhesive or the like, or to a metal tube by crimping, brazing, bonding, or other means known in the art.

Certain plastics, such as BioSpan® (segmented polyurethane), Bionate® (polycarbonate urethane), and Elasthane™ (polyetherurethane), manufactured by The Polymer Technology Group, Berkely, Calif., can be heat treated to resume an initially curved shape, after having been straightened a number of times. Such materials may be reinforced with nylon or stainless steel braiding or similar materials.

A burr or shaver may be used to erode, cut or shape tissue. Wires for delivering bipolar or monopolar radiofrequency (RF) energy for surgical cutting and coagulating tissue are known in the art. Optical fibers delivering laser energy, from, for instance, a pulsed Holmium: YAG laser emitting energy at 2.1 microns, may be used to vaporize bone or tissue or to coagulate or shrink tissue, or laser energy from a diode laser can be used to coagulate or shrink tissue, as is known in the art. High intensity incoherent light can coagulate tissue. Tissue can be killed by repetitive freezing and thawing, using cryotherapy devices known in the art. Tissue may also be cut or ablated by high pressure jets of water or saline, and focused ultrasound and microwave emitting devices which are also known in the art. These and other tissue-affecting means may be disposed within the distal end of such a tube.

If such a tube is passed through a plastic or metal sleeve or an instrument channel of an endoscope, provided the sleeve and endoscope are stiffer than the tube, the Nickel-titanium alloy distal end portion of the tube will conform to the shape of the sleeve or endoscope. However, when it emerges from the distal end of the sleeve or endoscope, it will resume its normal curved shape.

A proximal portion of the tube can bear markings, as for example: circles or half circles, at intervals of about 0.1 to 1 cm, and more preferably at intervals of about 0.5 cm, to enable a surgeon to determine the distance the tube has been extended from the distal end of the sleeve or endoscope and its angle of bend, even if the distal end of the tube has been inserted into tissue and cannot, itself, be seen.

The distal end portion of the tube can also bear such markings, enabling the surgeon to visually determine the extent to which the distal end portion of the tube has been extended from the endoscope and its angle of bend, even if the distal end of the tube has entered tissue and, again, cannot be seen.

For example, if the bent or curved portion of the tube is 2.5 cm in length and the angle of the bend is 90°, 0.5 cm equals one-fifth of the 2.5 cm length, which is one-fifth of the 90° bend or 18°. Accordingly, if the tube is extended 0.5 cm, its distal end will be bent at an angle of 18°. If it is extended 1 cm, its distal end will be bent at an angle of 36°, etc. Any combination of length and degree of bend can be employed to enable the device to provide a desired angle for effecting tissue removal, coagulation, shrinkage or other effects. The diameter of the heat treated bend of the tube is made to be less than the diameter of the space in which the device is to be deployed, to enable it to fit therein and be advanced to the target tissue.

The tissue effecting means can include a rotating burr or shaver, a source of monopolar or bipolar radiofrequency (RF) energy, a source of a cryotherapy fluid, a focused ultrasound or microwave emitter, a source of high pressure fluid or a source of laser or high intensity incoherent light energy. For example, an appropriate cutout or port in the distal end of the tube may expose the rotating blades of the burr or the guillotine of the shaver, and a suction or vacuum may be applied to a fluid channel in the tube to draw tissue into contact with the blades or burr to withdraw debris. A fluid channel through a tube containing an RF energy means can be used to infuse saline to create an electrically conductive environment or field. A fluid channel in the tube containing an optical fiber for delivery of laser energy can provide saline or water to cool the tissue. A gas, such as carbon dioxide, may be infused through the fluid channel to provide an environment through which laser energy at a wavelength of 2.1 microns, for example, may pass. Such energy would otherwise be highly absorbed by any intervening water or saline.

Alternatively, a passageway through a tube for delivery of laser, microwave, focused ultrasound or RF energy, or a space between the interior of a sleeve or sheath extending over the tube and the exterior of the tube, allows hot gasses from the vaporization of tissue to escape, avoiding excessive coagulation and edema. Alternatively, a suction or vacuum channel through the fluid channel in the tube may be employed to withdraw hot gases from the vaporization of tissue to avoid those same effects.

Mechanical instruments, such as a grasper, drill or reamer, as known in the art, may be employed, through the tube, as well.

Tissues that may be affected include, for example, bone, cartilage, the annulus fibrosus (tough outer layer) or nucleus pulposa (resilient center) of a spinal disc, the prostate gland, the tonsils, an ulcer, a blood clot, a tumor or the like. The tissue effect, for example, can range from cutting or vaporization at a temperature of about 100° C. or greater, coagulation from about 70° to 75° C., shrinkage from about 60° to 65° C. or denaturization from about 55° to 60° C.

Specifically, the present invention is a tissue affecting means consists of a tube with a distal end portion made of a superelastic, nickel/titanium memory metal alloy which has been heat-treated to retain a desired curved shape. The tube is moveably disposed within and constrained by a sleeve or by the instrument channel of an endoscope, both of which are stronger, that is stiffer than the distal end portion of the tube. When the distal end portion of the tube is extended from the distal end of the sleeve or endoscope, it returns to its curved shape. Markings about the proximal and/or distal end portion of the tube enable the operator to know to what extent the distal end portion of the tube has been extended out of the sleeve or endoscope and the angle of its curve or bend, even if the distal end of the tube has been inserted into tissue and cannot be seen.

The tissue affecting means can include a rotating burr, a shaver consisting of a rotating guillotine blade, in both cases exposed to tissue by a cut-out in the distal end of the tube, a monopolar or bipolar radiofrequency energy source, a source of laser energy, a mechanical tool or other forms of energy. The tube may be round or have any other cross-sectional shape, and its distal end can be blunt or, to facilitate penetration of tissue, can be sharply pointed or made in a syringe-like shape.

A primary objective of the present invention is to provide an apparatus and method of use of such apparatus that yields advantages not taught by the prior art.

Another objective is to provide such an invention capable of affecting tissue at a desired location hidden from view.

A further objective is to provide such an invention capable of drawing debris and fluids away from the affected tissue.

A still further objective is to provide such an invention capable of being inserted into a narrow passageway and then resuming its original shape.

A further objective is to provide such an invention capable of being extended by a known amount at a desired angle, although the tissue affecting means is not visible to an operator. Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the present invention. In such drawings:

FIG. 1A is a side elevational view of the present invention;

FIGS. 1B and 1C are cross-sectional side views of the distal end portion thereof as referenced in FIG. 1A by numeral 10; and

FIGS. 2-7 are similar views to that of FIG. 1B, showing alternate embodiments thereof.

### DETAILED DESCRIPTION OF THE INVENTION

The above described drawing figures illustrate the invention in at least one of its preferred embodiments, which is further defined in detail in the following description. Those having ordinary skill in the art may be able to make alterations and modifications in the present invention without departing from its spirit and scope. Therefore, it must be understood that the illustrated embodiments have been set forth only for the purposes of example and that they should not be taken as limiting the invention as defined in the following.

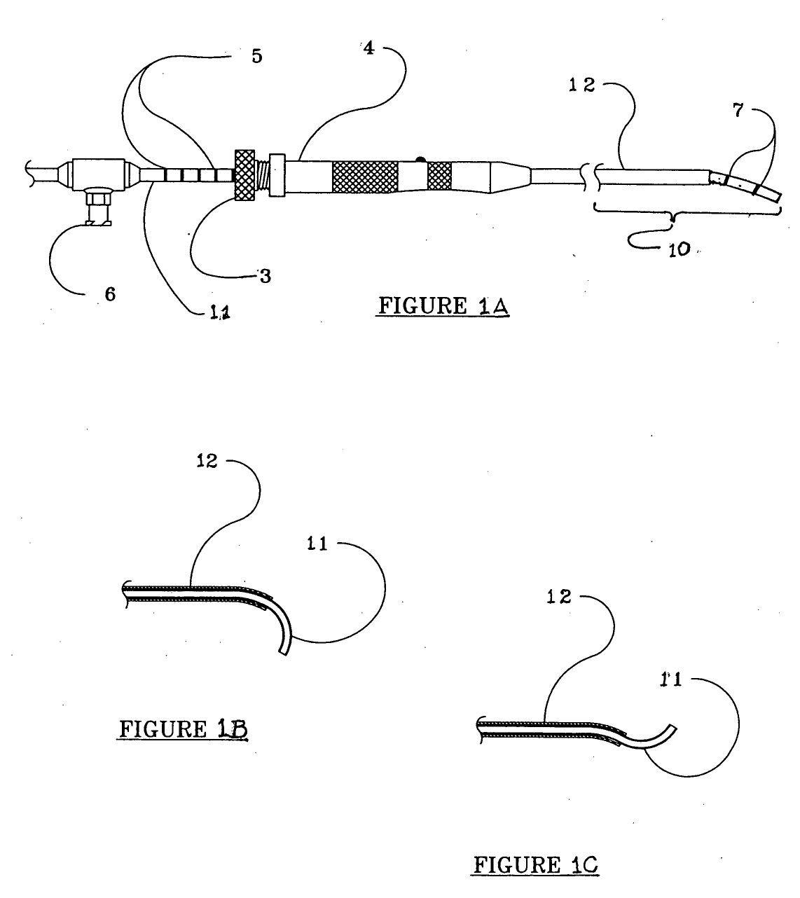

FIG. 1A illustrates the present invention with the distal portion identified by bracket 10. Relatively elastic member, tube 11, preferably has a fixed bend at its distal end, and is moveably extendable from relatively inelastic member, sleeve 12, which extends distally from handpiece 4. Tube 11 extends through gripping mechanism 3 in the proximal end of hand piece 4, and through handpiece 4 and sleeve 12, which enables convenient and easy use of the invention. When gripping mechanism 3 is turned clockwise, it engages and holds tube 11 in place within hand piece 4. For instance, gripping mechanism 3 can cause tubular tynes or a short length of soft tubing of a compressible material (not shown), to be compressed against tube 11, gripping it in place within hand piece 4. If gripping mechanism 3 is turned counter-clockwise and thereby loosened, tube 11 may be rotated until its bent portion is oriented so as to be brought into contact with a desired tissue.

Preferably, tube 11 contains markings 5 about its proximal end, for example, at intervals of about ½ cm, to enable the operator to ascertain the distance tube 11 has been extended out of sleeve 12 and its degree of curvature, so as to improve its ability to contact a target tissue. Luer fitting 6, which is in fluid communication with the interior of tube 11, enables a vacuum or suction to be drawn from or a liquid or gas to be infused through tube 11, as desired.

Sleeve 12 need not be straight. As seen in FIGS. 1B and 1C, the distal end portion of sleeve 12 may be bent, for example, as shown at an angle of about 30°, and the distal end portion of tube 11, may also be bent, for example, as shown at an angle of about 90°. Sleeve 12 and tube 11 may each be bent at any angle from about 10° to 90°, and preferably between about 20° and 60°.

Any number of combined angular deflections can be achieved by making a bend of up to 90° in the distal end portion of both tube 11 and sleeve 12 and rotating either tube 11 or sleeve 12 with respect to the other. This results in a total angulation of up to 180°. As shown, in FIGS. 1B and 1C, sleeve 12 is bent at an angle of about 30° and tube 11 is bent at an angle of about 90°, producing an angular deflection of 120° in FIGS. 1B and 600 in FIG. 1C. Tube 11 and sleeve 12 can each be bent at any desired angle to bring the distal end of tube 11 close to a desired tissue.

While sleeve 12 is shown as an independent element, it may also be an instrument channel of an endoscope, which typically is quite rigid and contains a compression means at its proximal end to removably fix and selectively position tube 11 within the instrument channel of the endoscope and prevent leakage of fluid therefrom.

As illustrated in FIG. 1A, in addition to markings 5 about the proximal end portion of tube 11, markings 7 may be placed at the distal end of tube 11. If tube 11 is inserted through the instrument channel of an endoscope, the surgeon can see the number of sections or intervals that the distal end portion of tube 11 has been advanced from the distal end of the endoscope and, therefore, determine the angle of its bend, even if some or all of the distal end portion of tube 11 has entered tissue and is not visible.

Referring now to the distal end portion 10 shown in FIG. 1A, and referring particularly to a first alternate embodiment thereof shown in FIG. 2, the distal end portion 10 of the device of the present invention consists of tube 11, preferably made of a superelastic Nickel-titanium alloy, as described above, that has been heat treated to retain the 90° bend shown. Clearly, alternate shapes would be further examples of such bends. The distal end of tube 11 is moveably extendable from the distal end of sleeve 12, which is substantially stiffer than tube 11 and therefore, not susceptible to being bent when tube 11 is present therein.

A burr 13 and its flexible means for rotation, which may be a braided wire, a wire rod, a hollow tube, a coiled spring, or similar means, as known in the art, are moveably disposed within tube 11. Optional bushing 15 supports rotating means 14 at the centerline of tube 11, and bushing 15 is perforated so as to allow debris, liquids, gasses, etc. to pass through it so as to move proximally within tube 11, and also to allow a suction to be drawn to move such materials through tube 11. In medical procedures, for instance, effectively significant portion of burr 13, at least 20% and more preferably, 33% or more of the surface of burr 13 is exposed by port 16 at the terminal portion of the distal end of tube 11.

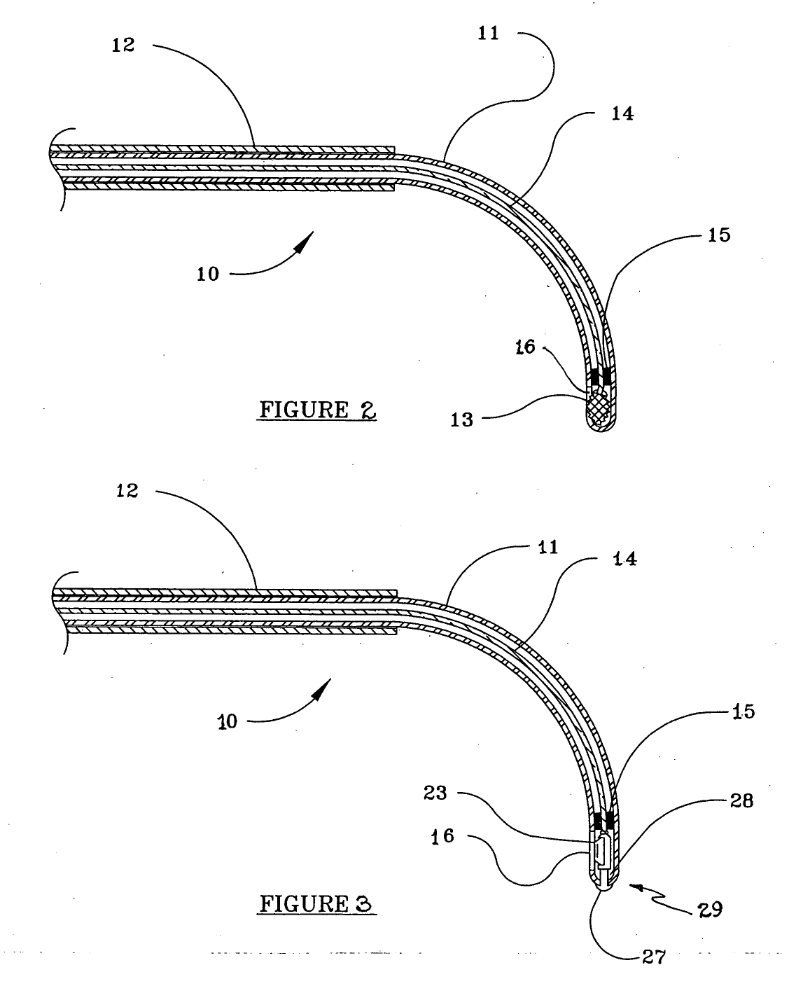

In an alternate embodiment, as seen in FIG. 3, the tissue affecting means may be a shaver, consisting of a semi-circular guillotine blade 23, as known in the art, which is driven by rotating means 14 as described above. Guillotine blade 23 is exposed by port 16 in the sidewall of the distal end of tube 11. A vacuum or suction may be applied through tube 11 and the open portion of bushing 15 to draw tissue into contact with guillotine blade 23 and to withdraw debris. Rotating means 14 may optionally terminate in cap 27, which rotatably extends through bore 28 in the terminal end 29 of tube 11.

In another alternate embodiment, as seen in FIG. 4, the distal end portion of tube 11 may contain insulated wires 33, for delivery of bipolar radiofrequency (RF) energy. Wires 33 extend from a source of bipolar RF energy, not shown, through tube 11 and insulation plug 34, which is fixedly attached by an adhesive and/or crimping within the terminal end of tube 11, and terminate in positive (+) and negative (−) electrodes 36 and 35, respectively. Insulation plug 34, like bushing 15, is perforated so as to allow debris, liquids, gasses, etc. to pass through it so as to move distally under pressure or proximally under suction through tube 11. Alternatively, if monopolar RF energy is desired, only one wire 33 and its positive (active) electrode are utilized, and a selectively larger negative electrode is attached to another area of the patient's body as an electrical return path. A multiplicity of RF electrodes may be employed to expand the tissue affecting area of the device.

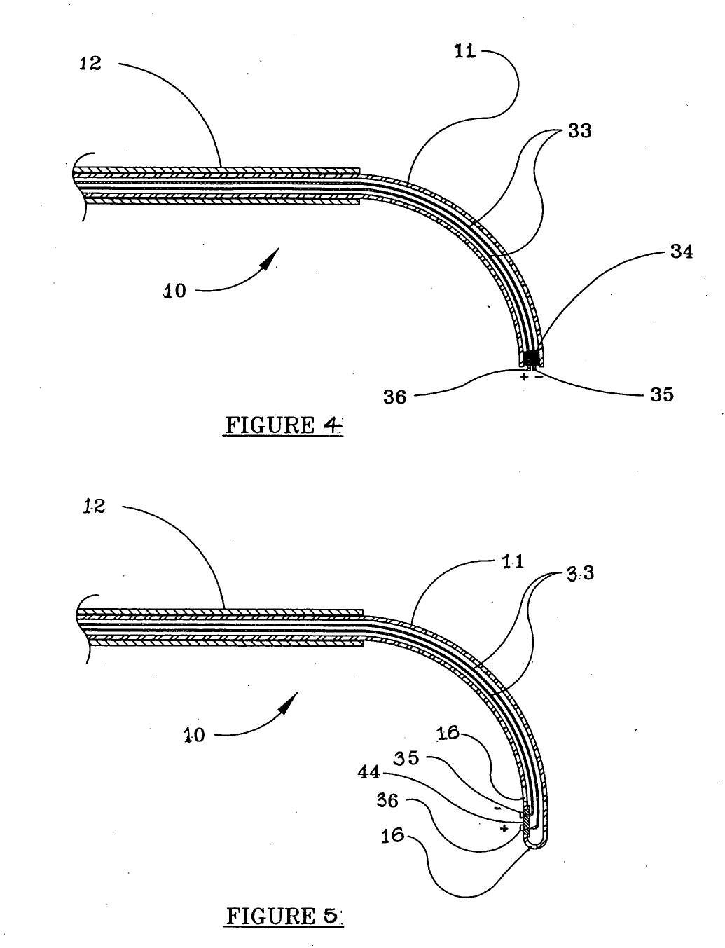

In another alternate embodiment similar to that of FIG. 4, as seen in FIG. 5, tube 11 conducts insulated wires 33 which terminate at positive (+) and negative (−) electrodes 36 and 35 respectively, held by an insulator 44 near the terminal end of tube 11. At least one port or opening 16 is formed in tube 11, as shown, or in insulator 44, to allow a fluid, such as electrically conductive saline, to be delivered by tube 11 to the area adjacent to electrodes 35 and 36 so as to create an electrically conductive field between electrodes 35, 36 and the tissue (not shown) that is in close proximity or in contact with electrodes 35, 36. RF electrodes 35 and 36 and insulator 44 are preferably positioned adjacent to port 16, or adjacent to an open terminal end of tube 11, such as shown in FIG. 4. A further port for a burr or shaver may be positioned distally or proximally to port 16. Such positioning provides enablement for coagulating any bleeding that occurs from the use of burr 13 of FIG. 2 or guillotine blade 23 of FIG. 3. Insulator 44 can also consist of small pieces of insulation surrounding the electrodes 35, 36 and attached by an adhesive or the like, within or atop openings (not separately shown) in tube 11.

In a further alternate embodiment, as shown in FIG. 6, tube 11 conducts optical fiber 53 from a source of laser energy (not separately shown). Such a source of laser energy may comprise, for example, a diode laser at a wavelength of between 610 and 980 nanometers, a Nd:YAG laser at a wavelength of 1,064 nanometers or, preferably, a Holmium:YAG laser at a wavelength of about 2,100 nanometers. Laser energy is used to cut, vaporize, coagulate, shrink or denature tissue. The direction of emission of the laser energy is indicated by dotted lines 54. Preferably, to prevent damage to optical fiber 53 from back-scatter of laser energy from tissue or stray emissions of laser energy, cylinder 55 is fixedly attached by an adhesive or other means, as known in the art, between the exterior of the distal end of optical fiber 53, from which the buffer coating 56 has been removed, and the interior of tube 11. A reflective outer surface of cylinder 55 reflects back-scattered energy and stray energy emissions from emission port 57. Therefore, cylinder 55 is preferably made of a material able to efficiently reflect laser energy of the wavelength being used. Such materials include silver, gold, copper foil and certain dielectric materials. High intensity incoherent light energy can also be used to coagulate or denature tissue.

Cylinder 55 may be semi-circular so as to provide an open portion for enabling a fluid to be infused through tube 11. A liquid, such as water or saline, may be infused to cool the targeted tissue and to flush debris away from the laser energy emitter. A biocompatible gas, such as carbon dioxide, can be infused to displace any intervening liquid, such as blood, plasma, interstitial fluids, water or saline, which, when present, absorb laser energy at wavelengths greater than 1800 nanometers. Infusion of a gas avoids the loss of energy that is required for vaporizing intervening liquids such as water or saline, both highly absorbent at wave lengths such as those between 1800 and 2200 nanometers, which is the range of Holmium laser energy.

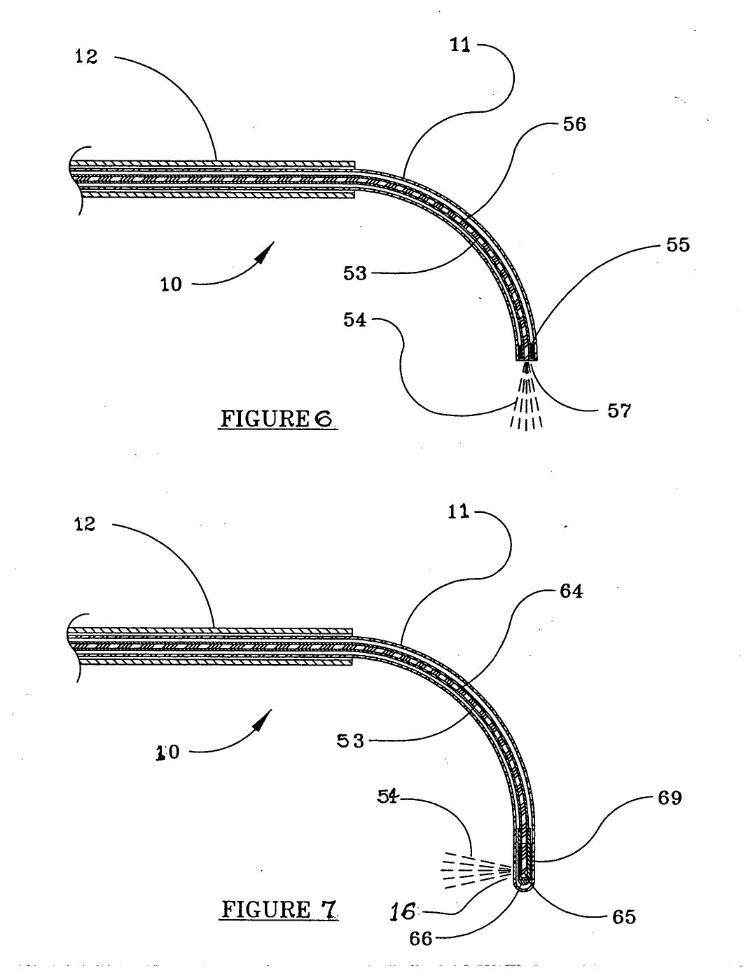

In another alternate embodiment, as shown in FIG. 7, the buffer coating 56 has been removed from the distal end portion of optical fiber 53, whose terminal end surface 65 has been beveled at an angle of 35° to 50°, and more preferably in the range from 38° to 42°. The bared distal portion of optical fiber 53 is encased by capillary tube 66, and attached thereto by an adhesive and/or thermal fusing process. Capillary tube 66 provides an air interface at beveled distal end surface 65, which enables total internal reflection of laser energy to occur, laterally at an angular range of between about 700 to 90° from the axis of optical fiber 53, and then out of port 16, as shown by dotted lines 54. Preferably, a semi-circular reflective insert 69 is disposed behind capillary tube 66 and extends over an angular range of from about 60° to about 270°, and more preferably from 90° to 240° of the rear, non-energy emitting exterior surface of capillary tube 66 to reflect any energy back-scattered from tissue or from beveled distal end 65, through capillary tube 66 and port 16. A liquid, such as water or saline, may be infused into tube 11 and pass over the energy emitting surface of capillary tube 66 to cool and flush out debris. Alternatively, a biocompatible gas, such as carbon dioxide, can be infused to displace any intervening liquid from the space between energy emitting surface of capillary tube 66 and the target tissue, enabling the laser energy to pass therethrough without significant loss.

The words used in this specification to describe the invention and its various embodiments are to be understood not only in the sense of their commonly defined meanings, but to include by special definition in this specification: structure, material or acts beyond the scope of the commonly defined meanings. Thus if an element can be understood in the context of this specification as including more than one meaning, then its use must be understood as being generic to all possible meanings supported by the specification and by the word or words describing the element.

The definitions of the words or elements of this described invention and its various embodiments are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the invention and its various embodiments or that a single element may be substituted for two or more elements in a claim.

Changes from the claimed subject matter as viewed by a person with ordinary skill in the art, now known or later devised, are expressly contemplated as being equivalents within the scope of the invention and its various embodiments. Therefore, obvious substitutions now or later known to one with ordinary skill in the art are defined to be within the scope of the defined elements. The invention and its various embodiments are thus to be understood to include what is specifically illustrated and described above, what is conceptually equivalent, what can be obviously substituted, and also what essentially incorporates the essential idea of the invention.

While the invention has been described with reference to at least one preferred embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

1. An apparatus comprising: a means for affecting tissue, the tissue affecting means disposed within a first enclosure having a preferred shape; the first enclosure disposed within a second enclosure and thereby constrained to the shape of the second enclosure, the second enclosure being more rigid than the first enclosure; the first enclosure free to translate within the second enclosure, thereby extending from the second enclosure to assume the preferred shape so as to move proximate a tissue surface.

2. The apparatus of claim 1 wherein the means for affecting tissue is at least one of a burr, a rotating guillotine, a source of fluid under elevated pressure, a cryogenic fluid, and a source of wave energy.

3. The apparatus of claim 1 wherein the first enclosure is a tube having a proximal end portion and a distal end portion.

4. The apparatus of claim 1 wherein the second enclosure is a constraining sleeve.

5. The apparatus of claim 3 wherein the distal end portion of the tube is made of a superelastic memory metal alloy treated to assume the preferred shape after being deformed therefrom.

6. The apparatus of claim 1 in which the tissue affecting means is a burr positioned adjacent a port in the first enclosure, thereby exposing an effectively significant portion of the burr exterior the first enclosure.

7. The apparatus of claim 6 in which the effectively significant portion is at least 20% of the burr exterior the first enclosure.

8. The apparatus of claim 1 in which the tissue affecting means is a rotating guillotine blade positioned adjacent a port in the relatively flexible enclosure.

9. The apparatus of claim 2 in which the source of wave energy is enabled for producing wave energy of, at least one of: bipolar radio frequency energy, monopolar radio frequency energy, incoherent light energy, focused ultrasound energy, microwave energy, and laser energy.

10. The apparatus of claim 5 in which the alloy contains about 55.8% of nickel and about 44.2% of titanium by weight.

11. The apparatus of claim 4 wherein the sleeve is an instrument channel of an endoscope.

12. The apparatus of claim 3 wherein the preferred shape of the tube encompasses a bend having a bend angle of at least 10°.

13. The apparatus of claim 3 wherein the distal end portion of the tube is heat-treated to maintain a curve having a selected degree of curvature.

14. The apparatus of claim 4 wherein one of the proximal end portion and distal end portion of the tube provides exterior markings for indicating extension of the tube from the sleeve.

15. The apparatus of claim 4 wherein the tube is free to rotate within the sleeve.

16. The apparatus of claim 3 wherein the tube contains a gas for displacing liquids and vapors exterior the distal end of the tube.

17. A method for affecting tissue comprising the steps of: inserting a first member, having a tendency to assume a preferred shape at a distal end thereof, into a second member; inserting a tissue affecting means into the first member; extending the distal end portion of the first member exterior the second member until the first member assumes the preferred shape thereby moving the tissue affecting means into proximity with a targeted tissue; and actuating the tissue affecting means so as to affect the targeted tissue selectively.

18. The method of claim 17 wherein the distal end is made of a superelastic memory metal alloy heat treated to assume the preferred shape.

19. The method of claim 17 further comprising the step of engaging the targeted tissue with the tissue affecting means when the targeted tissue is at least one of bone, cartilage, disc annulus fibrosus, disc nucleus pulposa, tumor, ulcer, tonsil, prostate gland, and clotted blood.

20. The method of claim 17 further comprising the step of applying a vacuum to the proximal end of the relatively elastic member thereby drawing away debris created at the tissue affecting means.

21. The method of claim 17 further comprising the step of applying a vacuum to the proximal end of the relatively elastic member thereby drawing the targeted tissue through a port in the relatively elastic member and into contact with one of a rotating guillotine blade and a burr.

22. The method of claim 17 further comprising the steps of delivering a fluid into the first member; and moving the fluid to at least one of the tissue affecting means and the targeted tissue.

23. The method of claim 17 further comprising the step of delivering an electrically conductive fluid into the first member thereby forming an electrically conductive field adjacent the targeted tissue.

24. The method of claim 17 further comprising the step of forming the first member as an instrument channel of an endoscope.

25. The method of claim 17 further comprising the stop of forming the tissue affecting means as at least one of a burr, a shaver, a cryogenic fluid source, a source of fluid under elevated pressure, a wave energy source, and a laser energy emitter.

26. The method of claim 17 wherein the selective tissue affecting is at least one of: cutting, vaporizing, ablating, abrading, freezing, coagulating, shrinking and denaturing.

27. The method of claim 17 further comprising the steps of marking at least one of a proximal end and a distal end of the first member; and controlling extension of the distal end of the first member from the second member by the marking.

28. The method of claim 17 further comprising the step of delivering a gas into the first member; and displacing liquids therewith exterior to the distal end of the member.

29. An apparatus for predictable lateral deflection comprising: a tissue affecting means disposed within a tube of a shape-retentive material; a distal end portion of the tube assuming a curved shape when not otherwise constrained; the tube movable within a constraining sleeve, the sleeve having a greater stiffness than the tube, wherein the distal end portion of the tube assumes the curved shape when extended from the sleeve.

30. The apparatus of claim 29 wherein at least the distal end portion of the tube is of a nickel-titanium alloy.

31. The apparatus of claim 29 wherein the tissue affecting means is at least one of a rotating burr, a moving guillotine blade, a radio frequency energy emitter, a microwave energy emitter, a focused ultrasound emitter, a source of cryogenic fluid, a source of fluid under elevated pressure, a source of incoherent light, and a laser energy emitter; and wherein the tissue affecting means is exposed for affecting tissue.

32. The apparatus of claim 30 wherein the nickel-titanium alloy contains about 55.8% of nickel and 44.2% of titanium by weight.

33. The apparatus of claim 29 wherein the distal end portion of the tube is heat treated to retain a selected curvature and radius.
